(19) United States
(12) Patent Application Publication
(10) Pub. No.: US 2007/0275029 A1
(43) Pub. Date: Nov. 29, 2007

(54) THERAPEUTIC DRUG COMBINATIONS AND DELIVERY SYSTEMS COMPRISING C-RAF KINASE ANTISENSE POLYNUCLEOTIDES FOR TREATING OCULAR DISEASES AND DISORDERS

(75) Inventors: John G. Clement, Delta (CA); Andrew J. Rae, Vancouver (CA)

Correspondence Address: SEED INTELLECTUAL PROPERTY LAW GROUP PLLC, 701 FIFTH AVE, SUITE 5400, SEATTLE, WA, 98104, US

(73) Assignee: ICO THERAPEUTICS INC., Vancouver (CA)

(21) Appl. No.: 11/753,900

(22) Filed: May 25, 2007

Related U.S. Application Data
(60) Provisional application No. 60/808,696, filed on May 26, 2006.

(51) Int. Cl. A61K 48/00; A61F 13/02

(52) U.S. Cl. 424/422; 514/44; 514/81

(57) ABSTRACT

The present invention is directed to combination therapies and drug delivery devices comprising antisense oligonucleotides directed to a raf gene. In certain embodiments, the combination therapies and drug delivery devices of the present invention are used to treat cancers and ocular diseases.

### CROSS-REFERENCE TO RELATED APPLICATION

This application claims the benefit under 35 U.S.C. § 119(e) of U.S. Provisional Patent Application No. 60/808,696 filed May 26, 2006, where this provisional application is incorporated herein by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 480231_{—}403_SEQUENCE_LISTING.txt. The text file is 9 KB, and created on May 25, 2007, and is being submitted electronically via EFS-Web, concurrent with the filing of the specification.

### BACKGROUND OF THE INVENTION

1. Field of the Invention

This invention relates to compositions and methods for modulating expression of raf genes, using antisense oligonucleotides directed to raf mRNAs. This invention is also directed to methods for inhibiting hyperproliferation of cells, and related methods of treating and preventing ocular diseases and cancer, using raf antisense oligonucleotides in combination with other therapeutic agents. Furthermore, this invention is directed to formulations and drug delivery devices that provide extended delivery of raf antisense oligonucleotides.

2. Description of the Related Art

Alterations in the cellular genes that directly or indirectly control cell growth and differentiation are considered to be the main cause of cancer. The raf gene family includes three highly conserved genes termed A-, B- and c-raf (also called raf-1). Raf genes encode protein kinases that are thought to play important regulatory roles in signal transduction processes that regulate cell proliferation. Expression of the c-raf protein is believed to play a role in abnormal cell proliferation since it has been reported that 60% of all lung carcinoma cell lines express unusually high levels of c-raf mRNA and protein. Rapp et al., The Oncogene Handbook, E. P. Reddy, A. M Skalka and T. Curran, eds., Elsevier Science Publishers, New York, 1988, pp. 213-253.

Antisense oligonucleotides targeting raf genes have been shown to inhibit raf gene expression and cell growth. In addition, antisense oligonucleotides have been demonstrated to have a therapeutic effect in a variety of animal models of disease, as well as human clinical trials. For example, antisense oligonucleotides targeting c-raf prevented or delayed allograft rejection in a murine vascularized heterotropic heart transplant model, and inhibited smooth muscle cell proliferation in both in vitro and in vivo models of atherosclerosis and restenosis following angioplasty. See, e.g., U.S. patent application Ser. No. 10/173,225. In addition, human clinical trials indicated that treatment with antisense oligonucleotides targeted to c-raf were therapeutically effective in the treatment of a variety of tumors, resulting in decreased tumor growth and metastasis. See, e.g., U.S. patent application Ser. No. 10/173,225.

Antisense oligonucleotides targeted to raf were also shown to reduce ocular neovascularization in a pig branch retinal vein occlusion model, supporting the use of these oligonucleotides to treat undesired angiogenesis or neovascularisation in the eye and other tissues and organs. Aberrant angiogenesis has been associated with a variety of ocular diseases and disorders, including, but not limited to, macular degeneration, diabetic retinopathy, and retinopathy of prematurity.

Macular degeneration affects between five and ten million patients in the United States, and it is the leading cause of blindness worldwide. Macular degeneration affects central vision and causes the loss of photoreceptor cells in the central part of retina called the macula. Macular degeneration can be classified into two types: dry type and wet type. The dry form is more common than the wet, with about 90% of age-related macular degeneration (ARMD) patients diagnosed with the dry form. The wet form of the disease and geographic atrophy, which is the end-stage phenotype of dry ARMD, lead to more serious vision loss. In wet ARMD, new blood vessels grow beneath the retina and leak blood and fluid. This leakage causes the retinal cells to die, creating blind spots in central vision. For the vast majority of patients who have the dry form of macular degeneration, no treatment is available. Because the dry form precedes development of the wet form of macular degeneration, intervention in disease progression of the dry form could benefit patients that presently have dry form and may delay or prevent development of the wet form.

Diabetic retinopathy occurs when diabetes damages blood vessels inside the retina. Non-proliferative retinopathy is a common, usually mild form that generally does not interfere with vision. Abnormalities are limited to the retina, and vision is impaired only if the macula is involved. However, if left untreated, it can progress to proliferative retinopathy, the more serious form of diabetic retinopathy. Proliferative retinopathy occurs when new blood vessels proliferate in and around the retina. Consequently, bleeding into the vitreous, swelling of the retina, and/or retinal detachment may occur, leading to blindness.

Unfortunately, the anatomical and physiological characteristics of the eye render retinal drug delivery a major challenge. Due to the presence of a formidable blood-retinal-barrier to solute transport, high doses of drugs are required to deliver therapeutic quantities of drug to the retina following systemic administration. Such large doses can lead to severe systemic side effects. Intravitreal injections are routinely used to circumvent the blood-retinal-barrier and to better deliver drugs to the retina. While this mode of administration provides significant drug concentrations in the retina with low doses, several retinal disorders require repeated intravitreal injections, which can result in retinal detachment, vitreal hemorrhage, and cataracts. Intravitreal implants capable of delivering drugs over a few months are currently available. However, these implants require surgical placement and removal, and the use of these implants has been associated with retinal detachment and endophthalmitis.

Clearly, there remains a long-felt need for improved compositions and methods for treating and preventing ocular diseases and cancers, as well as improved delivery devices and methods for delivering ocular drugs to the retina.

### BRIEF SUMMARY OF THE INVENTION

The present invention provides methods of regulating cell proliferation and neovascularization using a raf antisense oligonucleotide in combination with another drug. In addition, the present invention provides related compositions as well as drug delivery devices comprising a raf antisense oligonucleotide.

In one embodiment, the present invention includes a method of treating or preventing an ocular disease or disorder comprising administering to a patient or cells thereof a therapeutically effective amount of an oligonucleotide 8 to 50 nucleotides in length which is targeted to mRNA encoding human c-raf (SEQ ID NO: 64), wherein said oligonucleotide inhibits expression of human c-raf, in combination with an ocular agent. In particular embodiments, the ocular disease or disorder is age related macular degeneration, diabetic retinopathy, diabetic macular edema, cystoid macular edema, or corneal neovascularization. In various embodiments, the ocular agent is Macugen™, Avastin™, Sirna-027™, Cand5™, VEGF-TRAP, and Lucentis™, Visudyne™, Retaane™, Envizon™, Combretastatin™, AdPEDF, CAND5, kringle5, ganciclovir, ketotifen, verteporfin, pegaptanib, anecortare, dexamethasone, raibizumab, fluocinolone acetonide, or lerdelimumab.

In another embodiment, the present invention includes a method of treating or preventing growth or metastasis of a tumor comprising administering to a patient or cells thereof a therapeutically effective amount of an oligonucleotide 8 to 50 nucleotides in length which is targeted to mRNA encoding human c-raf (SEQ ID NO: 28), wherein said oligonucleotide inhibits expression of human c-raf, in combination with an antitumor agent. In particular embodiments, the tumor is an ovarian cancer. In one embodiment, the antitumor agent is a chemotherapeutic agent.

In a further embodiment, the present invention provides a drug delivery device comprising a biocompatible carrier and a therapeutically effective amount of an oligonucleotide 8 to 50 nucleotides in length which is targeted to mRNA encoding human c-raf (SEQ ID NO: 28), wherein said oligonucleotide inhibits expression of human c-raf. In one embodiment, the biocompatible carrier comprises a biocompatible matrix. In particular embodiments, the biocompatible matrix is selected from the group consisting of: a polymer, collagen, metal, hydroxyapatite, bioglass, aluminate, bioceramic materials, and purified proteins. In one embodiment, said biocompatible carrier comprises a microcapsule.

In various embodiments of the methods, compositions, and devices of the present invention, the oligonucleotide is a full phosphorothioate analog consisting of the sequence, TCCCGCCTGTGACATGCATT (SEQ ID NO:8), with 2′-O-methoxyethyl substitutions at positions 1-6 and 15-20, and wherein residues 7-14 are unmodified 2′-deoxy.

### DETAILED DESCRIPTION OF THE INVENTION

A. Therapeutic Compositions and Combinations

The present invention is based, in part, on the discovery of novel combinations of therapeutic agents that exhibit an enhanced ability to reduce cell proliferation and/or neovascularization. These combinations include a raf antisense oligonucleotide, which modulates expression of a raf gene, as well as one or more additional therapeutic agents, such as an ocular or cancer drug.

1. cRaf Antisense Oligonucleotides

The present invention employs oligonucleotides targeted to nucleic acids encoding raf, which modulate raf gene expression. Oligonucleotides and methods for modulation of c-raf (raf-1) and A-raf are presently preferred; however, compositions and methods for modulating expression of other forms of raf also have utility and are comprehended by this invention. Exemplary antisense raf oligonucleotides are described herein and also in U.S. Pat. Nos. 5,563,255, 5,952,229, 6,358,932, 5,656,612, 5,919,773, 6,410,518, and 6,806,258.

This relationship between an oligonucleotide and its complementary nucleic acid target to which it hybridizes is commonly referred to as “antisense.” “Targeting” an oligonucleotide to a chosen nucleic acid target, in the context of this invention, is a multistep process. The process usually begins with identifying a nucleic acid sequence whose function is to be modulated. This may be, as examples, a cellular gene (or mRNA made from the gene) whose expression is associated with a particular disease state, or a foreign nucleic acid from an infectious agent. In the present invention, the target is a nucleic acid encoding raf; in other words, the raf gene or mRNA expressed from the raf gene. The targeting process also includes determination of a site or sites within the nucleic acid sequence for the oligonucleotide interaction to occur such that the desired effect—modulation of gene expression—will result. Once the target site or sites have been identified, oligonucleotides are chosen which are sufficiently complementary to the target, i.e., hybridize sufficiently well and with sufficient specificity, to give the desired modulation.

In the context of this invention “modulation” means either inhibition or stimulation. Inhibition of abnormal raf gene expression is presently the preferred form of modulation. This modulation can be measured in ways which are routine in the art, for example by Northern blot assay of mRNA expression or Western blot assay of protein expression as taught in the examples of the instant application. Effects on cell proliferation or tumor cell growth can also be measured, as taught in the examples of the instant application. “Hybridization,” in the context of this invention, means hydrogen bonding, also known as Watson-Crick base pairing, between complementary bases, usually on opposite nucleic acid strands or two regions of a nucleic acid strand. Guanine and cytosine are examples of complementary bases which are known to form three hydrogen bonds between them. Adenine and thymine are examples of complementary bases which form two hydrogen bonds between them. “Specifically hybridizable” and “complementary” are terms which are used to indicate a sufficient degree of complementarity such that stable and specific binding occurs between the DNA or RNA target and the oligonucleotide. It is understood that an oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment, or, in the case of in vitro assays, under conditions in which the assays are conducted.

In preferred embodiments of this invention, oligonucleotides are provided which are targeted to mRNA encoding c-raf and A-raf. In accordance with this invention, persons of ordinary skill in the art will understand that mRNA includes not only the coding region which carries the information to encode a protein using the three letter genetic code, but also associated ribonucleotides which form a region known to such persons as the 5′-untranslated region, the 3′-untranslated region, the 5′ cap region, intron regions and intron/exon or splice junction ribonucleotides. Thus, oligonucleotides may be formulated in accordance with this invention that are targeted wholly or in part to these associated ribonucleotides as well as to the coding ribonucleotides. In preferred embodiments, the oligonucleotide is targeted to a translation initiation site (AUG codon) or sequences in the 5′- or 3′-untranslated region of the human c-raf mRNA. The functions of messenger RNA to be interfered with include all vital functions such as translocation of the RNA to the site for protein translation, actual translation of protein from the RNA, splicing or maturation of the RNA and possibly even independent catalytic activity which may be engaged in by the RNA. The overall effect of such interference with the RNA function is to cause interference with raf protein expression.

In the context of this invention, the term “oligonucleotide” refers to an oligomer or polymer of nucleotide or nucleoside monomers consisting of naturally occurring bases, sugars and intersugar (backbone) linkages. The term “oligonucleotide” also includes oligomers comprising non-naturally occurring monomers, or portions thereof, which function similarly. Such modified or substituted oligonucleotides are often preferred over native forms because of properties such as, for example, enhanced cellular uptake and increased stability in the presence of nucleases.

It is not necessary for all positions in a given compound to be uniformly modified, and in fact more than one of the aforementioned modifications may be incorporated in a single compound or even at a single nucleoside within an oligonucleotide. Certain preferred oligonucleotides of this invention are chimeric oligonucleotides. “Chimeric oligonucleotides” or “chimeras”, in the context of this invention, are oligonucleotides which contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the RNA target) and a region that is a substrate for RNase H cleavage. In one preferred embodiment, a chimeric oligonucleotide comprises at least one region modified to increase target binding affinity, and, usually, a region that acts as a substrate for RNAse H. Affinity of an oligonucleotide for its target (in this case a nucleic acid encoding raf) is routinely determined by measuring the Tm of an oligonucleotide/target pair, which is the temperature at which the oligonucleotide and target dissociate; dissociation is detected spectrophotometrically. The higher the Tm, the greater the affinity of the oligonucleotide for the target. In a more preferred embodiment, the region of the oligonucleotide which is modified to increase raf mRNA binding affinity comprises at least one nucleotide modified at the 2′ position of the sugar, most preferably a 2′-O-alkyl, 2′-O-alkyl-O-alkyl or 2′-fluoro-modified nucleotide. Such modifications are routinely incorporated into oligonucleotides and these oligonucleotides have been shown to have a higher Tm (i.e., higher target binding affinity) than 2′-deoxyoligonucleotides against a given target. The effect of such increased affinity is to greatly enhance antisense oligonucleotide inhibition of raf gene expression. RNAse H is a cellular endonuclease that cleaves the RNA strand of RNA:DNA duplexes; activation of this enzyme therefore results in cleavage of the RNA target, and thus can greatly enhance the efficiency of antisense inhibition. Cleavage of the RNA target can be routinely demonstrated by gel electrophoresis. In another preferred embodiment, the chimeric oligonucleotide is also modified to enhance nuclease resistance. Cells contain a variety of exo- and endo-nucleases which can degrade nucleic acids. A number of nucleotide and nucleoside modifications have been shown to make the oligonucleotide into which they are incorporated more resistant to nuclease digestion than the native oligodeoxynucleotide. Nuclease resistance is routinely measured by incubating oligonucleotides with cellular extracts or isolated nuclease solutions and measuring the extent of intact oligonucleotide remaining over time, usually by gel electrophoresis. Oligonucleotides which have been modified to enhance their nuclease resistance survive intact for a longer time than unmodified oligonucleotides. A variety of oligonucleotide modifications have been demonstrated to enhance or confer nuclease resistance. Oligonucleotides which contain at least one phosphorothioate modification are presently more preferred. In some cases, oligonucleotide modifications which enhance target binding affinity are also, independently, able to enhance nuclease resistance.

The oligonucleotides in accordance with this invention preferably are from about 8 to about 50 nucleotides in length. In the context of this invention it is understood that this encompasses non-naturally occurring oligomers as hereinbefore described, having 8 to 50 monomers. Particularly preferred are antisense oligonucleotides comprising from about 8 to about 30 nucleobases (i.e. from about 8 to about 30 linked nucleosides).

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotri-esters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH.sub.2 component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262. Further teaching of PNA compounds can be found in Nielsen et al. (Science, 1991, 254, 1497-1500).

Most preferred embodiments of the invention are oligonucleotides with phosphorothioate backbones and oligonucleosides with heteroatom backbones, and in particular —CH.sub.2-NH—O—CH.sub.2-, —CH.sub.2-N(CH.sub.3)-O—CH.sub.2-[known as a methylene (methylimino) or MMI backbone], —CH.sub.2-O—N(CH.sub.3)-CH.sub.2-, —CH.sub.2-N(CH.sub.3)-N(CH.sub.3)-CH.sub.2- and —O—N(CH.sub.3)-CH.sub.2-CH.sub.2-[wherein the native phosphodiester backbone is represented as —O—P—O—CH.sub.2-] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O—, S—, or N-alkyl, O-alkyl-O-alkyl, O—, S—, or N-alkenyl, or O—, S— or N-alkynyl, wherein the alkyl, alkenyl and alkynyl may be substituted or unsubstituted C.sub.1 to C.sub.10 alkyl or C2 to C10 alkenyl and alkynyl. Particularly preferred are O[(CH.sub.2).sub.nO].sub.mCH.sub.3, O(CH.sub.2).sub.nOCH.sub.3, O(CH.sub.2).sub.2ON(CH.sub.3).sub.2, O(CH.sub.2).sub.nNH.sub.2, O(CH.sub.2).sub.nCH.sub.3, O(CH.sub.2).sub.nONH.sub.2, and O(CH.sub.2).sub.nON[(CH.sub.2).sub.nCH.sub.3)].sub.2, where n and m are from 1 to about 10. Other preferred oligonucleotides comprise one of the following at the 2′ position: C.sub.1 to C.sub.10 lower alkyl, substituted lower alkyl, alkaryl, aralkyl, O-alkaryl or O-aralkyl, SH, SCH.sub.3, OCN, Cl, Br, CN, CF.sub.3, OCF.sub.3, SOCH.sub.3, SO.sub.2CH.sub.3, ONO.sub.2, NO.sub.2, N.sub.3, NH.sub.2, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH.sub.2CH.sub.2OCH.sub.3, also known as 2′-O-(2-methoxyethyl) or 2′-MOE) (Martin et al., Helv. Chim. Acta 1995, 78, 486-504) i.e., an alkoxyalkoxy group. Further preferred modifications include 2′-dimethylaminooxyethoxy, i.e., a O(CH.sub.2).sub.20N(CH.sub.3).sub.2 group, also known as 2′-DMAOE, and 2′-dimethylaminoethoxyethoxy (2′-DMAEOE) as described in examples herein below.

Other preferred modifications include 2′-methoxy (2′-O—CH.sub.3), 2′-aminopropoxy (2′-OCH.sub.2CH.sub.2CH.sub.2NH.sub.2) and 2′-fluoro (2′-F) Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugars structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C or m5c), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808, those disclosed in the Concise Encyclopedia Of Polymer Science And Engineering 1990, pages 858-859, Kroschwitz, J. I., ed. John Wiley & Sons, those disclosed by Englisch et al. (Angewandte Chemie, International Edition 1991, 30, 613-722), and those disclosed by Sanghvi, Y. S., Chapter 15, Antisense Research and Applications 1993, pages 289-302, Crooke, S. T. and Lebleu, B., ed., CRC Press. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2.degree. C. (Sanghvi, Y. S., Crooke, S. T. and Lebleu, B., eds., Antisense Research and Applications 1993, CRC Press, Boca Raton, pages 276-278) and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941.

Another modification of the oligonucleotides of the invention involves chemically linking to the oligonucleotide one or more moieties or conjugates which enhance the activity, cellular distribution or cellular uptake of the oligonucleotide. Such moieties include but are not limited to lipid moieties such as a cholesterol moiety (Letsinger et al., Proc. Natl. Acad. Sci. USA 1989, 86, 6553-6556), cholic acid (Manoharan et al., Bioorg. Med. Chem. Lett. 1994, 4, 1053-1059), a thioether, e.g., hexyl-5-tritylthiol (Manoharan et al., Ann. N.Y. Acad. Sci. 1992, 660, 306-309; Manoharan et al., Bioorg. Med. Chem. Let. 1993, 3, 2765-2770), a thiocholesterol (Oberhauser et al., Nucl. Acids Res. 1992, 20, 533-538), an aliphatic chain, e.g., dodecandiol or undecyl residues (Saison-Behmoaras et al., EMBO J. 1991, 10, 1111-1118; Kabanov et al., FEBS Lett. 1990, 259, 327-330; Svinarchuk et al., Biochimie 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett. 1995, 36, 3651-3654; Shea et al., Nucl. Acids Res. 1990, 18, 3777-3783), a polyamine or a polyethylene glycol chain (Manoharan et al., Nucleosides & Nucleotides 1995, 14, 969-973), or adamantane acetic acid (Manoharan et al., Tetrahedron Lett. 1995, 36, 3651-3654), a palmityl moiety (Mishra et al., Biochim. Biophys. Acta 1995, 1264, 229-237), or an octadecylamine or hexylamino-carbonyl-oxycholesterol moiety (Crooke et al., J. Pharmacol. Exp. Ther., 1996, 277, 923-937).

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

The oligonucleotides used in accordance with this invention may be conveniently and routinely made through the well-known technique of solid phase synthesis. Equipment for such synthesis is sold by several vendors including Applied Biosystems. Any other means for such synthesis may also be employed; the actual synthesis of the oligonucleotides is well within the talents of the routineer. It is also well known to use similar techniques to prepare other oligonucleotides such as the phosphorothioates and alkylated derivatives. It is also well known to use similar techniques and commercially available modified amidites and controlled-pore glass (CPG) products such as biotin, fluorescein, acridine or psoralen-modified amidites and/or CPG (available from Glen Research, Sterling Va.) to synthesize fluorescently labeled, biotinylated or other modified oligonucleotides such as cholesterol-modified oligonucleotides.

The raf antisense oligonucleotides of the present invention modulate raf gene expression. In certain embodiments, they inhibit c-raf gene expression. The sequences of exemplary c-raf antisense oligonucleotides are shown in Table 1.

TABLE 1
Human c-raf Kinase Antisense Oligonucleotides

| | | |
|---|---|---|
| | | SEQ |
| | | ID |
| Sequence (5′-3′) | Site | NO: |

| | | |
|---|---|---|
| TGAAGGTGAGCTGGAGCCAT | Coding | 1 |
| GCTCCATTGATGCAGCTTAA | AUG | 2 |
| CCCTGTATGTGCTCCATTGA | AUG | 3 |
| GGTGCAAAGTCAACTAGAAG | STOP | 4 |
| ATTCTTAAACCTGAGGGAGC | 5′UTR | 5 |
| GATGCAGCTTAAACAATTCT | 5′UTR | 6 |
| CAGCACTGCAAATGGCTTCC | 3′UTR | 7 |
| TCCCGCCTGTGACATGCATT | 3′UTR | 8 |
| GCCGAGTGCCTTGCCTGGAA | 3′UTR | 9 |
| AGAGATGCAGCTGGAGCCAT | Coding | 10 |
| AGGTGAAGGCCTGGAGCCAT | Coding | 11 |
| GTCTGGCGCTGCACCACTCT | 3′UTR | 12 |
| CTGATTTCCAAAATCCCATG | 3′UTR | 13 |
| CTGGGCTGTTTGGTGCCTTA | 3′UTR | 14 |
| TCAGGGCTGGACTGCCTGCT | 3′UTR | 15 |
| GGTGAGGGAGCGGGAGGCGG | 5′UTR | 16 |
| CGCTCCTCCTCCCCGCGGCG | 5′UTR | 17 |
| TTCGGCGGCAGCTTCTCGCC | 5′UTR | 18 |
| GCCGCCCCAACGTCCTGTCG | 5′UTR | 19 |
| TCCTCCTCCCCGCGGCGGGT | 5′UTR | 20 |
| CTCGCCCGCTCCTCCTCCCC | 5′UTR | 21 |
| CTGGCTTCTCCTCCTCCCCT | 3′UTR | 22 |
| CGGGAGGCGGTCACATTCGG | 5′UTR | 23 |
| TCTGGCGCTGCACCACTCTC | 3′UTR | 24 |

Exemplary 2′-modified c-raf antisense oligonucleotides comprising either phosphodiester (P.dbd.O) or phosphorothioate (P.dbd.S) backbones and uniformly substituted at the 2′ position of the sugar with either a 2′-O-methyl, 2′-O-propyl, or 2′-fluoro group are shown in Table 2.

TABLE 2

Uniformly 2′ Sugar-modified c-raf
Oligonucleotides

| | | | |
|---|---|---|---|
| | | | SEQ |
| | | | ID |
| Sequence | Site | Motif | NO. |

| | | | |
|---|---|---|---|
| TCCCGCCTGTGACATGCATT | 3′UTR | OMe/P = S | 8 |
| CGGGAGGCGGTCACATTCGG | 5′UTR | OMe/P = S | 23 |
| GGTGAGGGAGCGGGAGGCGG | 5′UTR | OMe/P = S | 16 |
| CGCTCCTCCTCCCCGCGGCG | 5′UTR | OMe/P = S | 17 |
| TTCGGCGGCAGCTTCTCGCC | 5′UTR | OMe/P = S | 18 |
| GCCGCCCCAACGTCCTGTCG | 5′UTR | OMe/P = S | 19 |
| ATTCTTAAACCTGAGGGAGC | 5′UTR | OMe/P = S | 5 |
| GATGCAGCTTAAACAATTCT | 5′UTR | OMe/P = S | 6 |
| GCTCCATTGATGCAGCTTAA | AUG | OMe/P = S | 2 |
| CCCTGTATGTGCTCCATTGA | AUG | OMe/P = S | 3 |
| CGGGAGGCGGTCACATTCGG | 5′UTR | OPr/P = O | 23 |
| GGTGAGGGAGCGGGAGGCGG | 5′UTR | OPr/P = O | 16 |
| CGCTCCTCCTCCCCGCGGCG | 5′UTR | OPr/P = O | 17 |
| TTCGGCGGCAGCTTCTCGCC | 5′UTR | OPr/P = O | 18 |
| GCCGCCCCAACGTCCTGTCG | 5′UTR | OPr/P = O | 19 |
| ATTCTTAAACCTGAGGGAGC | 5′UTR | OPr/P = O | 5 |
| GATGCAGCTTAAACAATTCT | 5′UTR | OPr/P = O | 6 |
| GCTCCATTGATGCAGCTTAA | AUG | OPr/P = O | 2 |
| CCCTGTATGTGCTCCATTCA | AUG | OPr/P = O | 3 |
| CGGGAGGCGGTCACATTCGG | 5′UTR | 2′F/P = S | 23 |

Exemplary chimeric oligonucleotides having SEQ ID NO: 8 and having central “gap” regions of 6, 8, or 10 deoxynucleotides flanked by two regions of 2′-O-methyl modified nucleotides are shown in Table 3. Backbones were uniformly phosphorothioate. Additional chimeric oligonucleotides having one or more regions of 2′-O-methyl modification and uniform phosphorothioate backbones are shown in Table 3. All are phosphorothioates; bold regions indicate 2′-O-methyl modified regions.

TABLE 3

Chimeric 2′-O-methyl P = S c-raf
oligonucleotides

| | | SEQ |
|---|---|---|
| | Target | ID |
| Sequence | site | NO: |
| TCCTCCTCCCCGCGGCGGGT | 5′UTR | 20 |
| TCCTCCTCCCCGCGGCGGGT | 5′UTR | 20 |
| CTCGCCCGCTCCTCCTCCCC | 5′UTR | 21 |
| CTCGCCCGCTCCTCCTCCCC | 5′UTR | 21 |
| TTCTCGCCCGCTCCTCCTCC | 5′UTR | 25 |
| TTCTCGCCCGCTCCTCCTCC | 5′UTR | 25 |
| TTCTCCTCCTCCCCTGGCAG | 3′UTR | 26 |
| CTGGCTTCTCCTCCTCCCCT | 3′UTR | 22 |
| CTGGCTTCTCCTCCTCCCCT | 3′UTR | 22 |
| CCTGCTGGCTTCTCCTCCTC | 3′UTR | 27 |

Additional exemplary chimeric oligonucleotides with various 2′ modifications are shown in Table 4. All are phosphorothioates; bold regions indicate 2′-modified regions.

TABLE 4

Chimeric 2′-modified P = S c-raf
oligonucleotides

| | | | |
|---|---|---|---|
| | | | SEQ |
| | Target | | ID |
| SEQUENCE | site | Modification | NO: |

| | | | |
|---|---|---|---|
| TCCCGCCTGTGACATGCATT | 3′UTR | 2′-O-Me | 8 |
| TCCCGCCTGTGACATGCATT | 3′UTR | 2′-O-Me | 8 |
| TCCCGCCTGTGACATGCATT | 3′UTR | 2′-O-Me | 8 |
| TCTGGCGCTGCACCACTCTC | 3′UTR | 2′-O-Me | 24 |
| TCCCGCCTGTGACATGCATT | 3′UTR | 2′-O-Pro | 8 |
| TCCCGCCTGTGACATGCATT | 3′UTR | 2′-F | 8 |
| TCTGGCGCTGCACCACTCTC | 3′UTR | 2′-F | 24 |

Exemplary chimeric oligonucleotides with 2′-O-propyl sugar modifications and chimeric P═O/P═S backbones are shown in Table 5, in which italic regions indicate regions which are both 2′-modified and have phosphodiester backbones.

TABLE 5

Chimeric 2′-modified P = S/P = O
c-raf oligonucleotides

| | | | |
|---|---|---|---|
| | | | SEQ |
| | | | ID |
| Target Sequence | site | Modification | NO: |

| | | | |
|---|---|---|---|
| TCCCGCCTGTGACATGCATT | 3′UTR | 2′-O-Pro | 8 |
| TCTGGCGCTGCACCACTCTC | 3′UTR | 2′-O-Pro | 24 |

In one embodiment, a c-raf antisense oligonucleotide is a full phosphorothioate analog consisting of the sequence, TCCCGCCTGTGACATGCATT (SEQ ID NO:8), with 2′-O-methoxyethyl substitutions at positions 1-6 and 15-20, and wherein residues 7-14 are unmodified 2′-deoxy.

The present invention contemplates the use of any and all raf antisense oligonucleotides comprising or consisting of one or more of these exemplified sequences, in addition to any other raf antisense oligonucleotide capable of modulating raf gene expression. In particular embodiments, a raf antisense oligonucleotide of the present invention selectively modulates expression of one raf gene, while in other embodiments, a raf antisense oligonucleotide of the present invention modulates expression of two or more raf genes.

2. Other Therapeutic Agents

The present invention provide compositions and methods comprising or involving the delivery of a raf antisense oligonucleotide in combination with another therapeutic agent. The present invention contemplates the use of a wide variety of therapeutic agents suitable for treating various diseases and disorders, including, e.g., ocular drugs, cancer drugs, anti-angiogenic and immunosuppressive agents.

a. Ocular Drugs

In certain embodiments, the present invention includes compositions, formulations, and methods directed to combinations of an oligonucleotide of the invention in conjunction with an ocular drug. Ocular drugs include a variety of different types of molecules, including peptides, polypeptides, polynucleotides, antibodies, small organic compounds, metals, small inorganic molecules and radionuclides.

In particular embodiments, the ocular drug is an antiangiogenic factor, such as an inhibitor of vascular endothelial growth factor, e.g., Macugen™, Avastin™, Sirna-027™, Cand5™, VEGF-TRAP, Envison™ and Lucentis™. A variety of different types of agents directed against VEGF are used in combination therapies according to the invention, including, e.g., antisense, siRNAs, ribozymes, antibodies, shRNAs, VEGF tyrosine kinase inhibitors, Src kinase inhibitors and photodynamic therapy such as verteporfin (Visudyne™), Photofrin™ and texaphyrins. Other ocular drugs useful in combination with a raf antisense oligonucleotide according to methods of the present invention include, e.g., Visudyne™, Retaane™, Envizon™, Combretastatin™, AdPEDF, CAND5, kringle5, ganciclovir, ketotifen, verteporfin, pegaptanib, anecortave acetate, dexamethasone, ranibizumab, fluocinolone acetonide, and lerdelimumab. Other representative ocular drugs that may be used according to the present invention include, but are not limited to, those described in Bartlett, J. D and Jaanus, S., General Opthamology, Clinicla Ocular Pharmacology, (Butterwoth-Heinemann, 1995) and Hom, M. M., Mosby's Ocular Drug Consult.

b. Cancer Drugs

In certain embodiments, the present invention includes compositions, formulations and methods directed to combinations of an oligonucleotide of the invention in conjunction with a cancer drug, i.e., an anti-tumor agent. Cancer drugs include a variety of different types of molecules, including peptides, polypeptides, polynucleotides, antibodies, small organic compounds, metals, small inorganic molecules and radionuclides.

In particular embodiments, the cancer drug is tumor necrosis factor (TNF), an antibody capable of inhibiting or neutralizing the angiogenic activity of acidic or basic fibroblast growth factor (bFGF) or hepatocyte growth factor (HGF), an antibody capable of inhibiting or neutralizing the coagulant activities of tissue factor, protein C, or protein S (see Esmon, et al., PCT Patent Publication No. WO 91/01753, published Feb. 21, 1991), or an antibody capable of binding to HER2 receptor (see Hudziak, et al., PCT Patent Publication No. WO 89/06692, published Jul. 27, 1989) and a vascular damaging agent such as CA4P.

In other embodiments, an additional therapeutic agent is an inhibitor of angiogenesis. Examples of such inhibitors include, but are not limited to, Angioarrestin, Angiostatin (plasminogen fragment), Antiangiogenic antithrombin III, Cartilage-derived inhibitor (CDI), CD59 complement fragment, Endostatin (collagen XVIII fragment), Fibronectin fragment, Gro-beta, Heparinases, Heparin hexasaccharide fragment, Human chorionic gonadotropin (hCG), Interferon alpha/beta/gamma, Interferon inducible protein (IP-10), Interleukin-12, Kringle 5 (plasminogen fragment), Metalloproteinase inhibitors (TIMPs), 2-Methoxyestradiol. Placental ribonuclease inhibitor, Plasminogen activator inhibitor, Platelet factor-4 (PF4). Prolactin 16 kD fragment, Proliferin-related protein (PRP), Retinoids, Tetrahydrocortisol-S, Thrombospondin-1 (TSP-1), Transforming growth factor-beta (TGF-b), Vasculostatin, and Vasostatin (calreticulin fragment).

In other embodiments, an additional therapeutic agent is an inhibitor of complement H activity.

In other embodiments, an additional therapeutic agent is an inhibitor of CXCR4 such as AMD3100 and AMD8664 or a monoclonal antibody or antibody fragment or aptamer that interacts with stromal derived factor-1 (SDF-1).

In other embodiments, an additional therapeutic agent is an inhibitor of CCR3 or anti-eotaxin agent such as CAT-213.

Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhne-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid derivatives such as Targretin™ (bexarotene), Panretin™ (alitretinoin); ONTAK™ (denileukin diftitox); esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

B. Pharmaceutical Compositions

The present invention further provides pharmaceutical compositions and formulations comprising one or more therapeutic agents of the invention. Raf antisense oligonucleotides of the present invention may be administered in combination with a second therapeutic agent for a variety of reasons, including increased efficacy or to reduce undesirable side effects. The raf antisense oligonucleotide may be administered prior to, subsequent to, or simultaneously with the additional therapeutic agent. The raf antisense oligonucleotide may be delivered in a separate formulation or in the same formulation as the additional chemotherapeutic agent(s). Furthermore, where a raf antisense oligonucleotide of the present invention is administered in a drug delivery device, including those described herein, the second therapeutic agent may be delivered independently or also be included in the drug delivery device. In a preferred embodiment, raf antisense oligonucleotides of the present invention are administered in combination with an additional that provides an increased or synergistic improvement in tumor reduction based on mechanism of action and non-overlapping toxicity profiles.

Therapeutic agents of the invention may be formulated as pharmaceutical compositions suitable for delivery to a subject. The pharmaceutical compositions of the invention will often further comprise one or more buffers (e.g., neutral buffered saline or phosphate buffered saline), carbohydrates (e.g., glucose, mannose, sucrose, dextrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate.

Suitable formulations for use in the present invention can be found, e.g., in Remington's Pharmaceutical Sciences, Mack Publishing Company, Philadelphia, Pa., 17^{th} Ed. (1985). Often, intravenous compositions will comprise a solution of the therapeutic agent in an acceptable carrier, such as an aqueous carrier. Any of a variety of aqueous carriers can be used, e.g., water, buffered water, 0.4% saline, 0.9% isotonic saline, 0.3% glycine, 5% dextrose, and the like, and may include glycoproteins for enhanced stability, such as albumin, lipoprotein, globulin, etc. Often, normal buffered saline (135-150 mM NaCl) or 5% dextrose will be used. These compositions can be sterilized by conventional sterilization techniques, such as filtration. The resulting aqueous solutions may be packaged for use or filtered under aseptic conditions and lyophilized, the lyophilized preparation being combined with a sterile aqueous solution prior to administration. The compositions may also contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride calcium chloride, etc.

Therapeutic agents and formulations thereof are administered in any of a number of ways, including parenteral, intravenous, systemic, local, oral, intratumoral, intramuscular, intraocular, eye drops, subcutaneous, intraperitoneal, inhalation, or any such method of delivery.

In one embodiment, the therapeutic agents of the present invention are administered parenterally, i.e., intraarticularly, intravenously, intraperitoneally, subcutaneously, or intramuscularly. In a specific embodiment, they are administered by intravenous infusion or intraperitoneally by a bolus injection. For example, in one embodiment, a patient is given an intravenous infusion of one or more therapeutic agents through a running intravenous line over, e.g., 5-10 minutes, 15-20 minutes, 30 minutes, 60 minutes, 90 minutes, or longer. In one embodiment, a 60 minute infusion is used. In other embodiments, an infusion ranging from 6-10 or 15-20 minutes is used. Such infusions can be given periodically, e.g., once every 1, 3, 5, 7, 10, 14, 21, or 28 days or longer, preferably once every 7-21 days, and preferably once every 7 or 14 days.

In another embodiment, the therapeutic agents of the present invention are administered to the eye. The human eye can be divided into the anterior and posterior anatomical segments. Drug delivery to the anterior segment is primarily achieved through topical application, and significant success has been achieved in delivering drugs to this area. However, the delivery of drugs to the posterior segment of the eye poses a great challenge. Currently, the posterior segment disease treatment focuses on four approaches to deliver drugs—topical, systemic, intraocular, and periocular, including subconjunctival, subtenon, and retrobulbar modes of administration.

Topical application of drugs for treatment of posterior eye disorders is not very effective due to the long diffusional path length, rapid precorneal elimination due to solution drainage, normal or induced lacrimation, and corneal epithelial impermeability to molecules larger than 5 kDa. Although the systemic approach can deliver drugs to the eye, systemically administered drugs have poor access to the eye tissues because of the blood-aqueous barrier (which prevents the substances from entering into the aqueous humor) and because of the blood-retinal barrier (which severely limits drug entry into the extravascular space of the retina and into the vitreous). Consequently, large systemic doses are required, and this can induce toxicity and unwanted side effects. Intravitreal injections are an effective way of delivering drugs to the vitreoretinal region. However, intravitreal injections can potentially induce retinal detachment, hemorrhage, endophthalmitis, and cataracts.

Periocular modes of administration include subconjunctival, subtenon, and retrobulbar. In all of these modes, the drug is interfaced with sclera. There is substantial evidence indicating that drugs administered subconjunctivally can reach the vitreous effectively. The sclera does not provide an effective barrier to the entry of drugs, and even solutes of relatively large molecular weight can penetrate through it. The drugs can gain entry into the posterior segments from the subconjunctival space after entering the sclera. Systemic absorption is low with subconjunctival route, which can lower systemic side effects while providing a localized drug effect.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Formulations for oral administration may include powders or granules, suspensions or solutions in water or non-aqueous media, capsules, sachets, or tablets. Thickeners, flavorings, diluents, emulsifiers, dispersing aids or binders may be desirable.

Formulations for parenteral administration may include sterile aqueous solutions which may also contain buffers, diluents and other suitable additives.

In addition to such pharmaceutical carriers, cationic lipids may be included in the formulations to facilitate oligonucleotide uptake. One such composition shown to facilitate uptake is LIPOFECTIN (a 1:1 liposome formulation of the cationic lipid N-[1-(2,3-dioleyloxy)propyl]-N,N,N-trimethylammonium chloride (DOTMA) and dioleoyl phosphatidylethanolamine (DOPE)) (BRL, Bethesda Md.).

Dosing is generally dependent on severity and responsiveness of the condition to be treated, with course of treatment lasting from several days to several months or until a cure is effected or a diminution of disease state is achieved. Optimal dosing schedules can be calculated from measurements of drug accumulation in the body or at a localized site or based upon a patient's response. Persons of ordinary skill can easily determine optimum dosages, dosing methodologies, and repetition rates. Optimum dosages may vary depending on the relative potency of individual oligonucleotides, and can generally be estimated based on EC50's in in vitro and in vivo animal studies.

Routes of administration and dosages can be readily determined by the skilled artisan upon considering the particular disease and, if present, drug delivery device used. In one embodiment, administration comprises intravitreal injection in a drug delivery system that will extend the duration of action of a raf antisense oligonucleotide for treatment intervals between injections of at least 3 months, ideally at least 6 months or more between injections. The drug delivery system will extend the ocular pharmacokinetics (half-life) of a raf antisense oligonucleotide beyond that obtained by using simply the 2-o′-MOE substitution of the second generation antisense molecule.

The formulation of therapeutic compositions and their subsequent administration is within the skill in the art. In general, for therapeutics, a patient suspected of needing such therapy is given an oligonucleotide in accordance with the invention, commonly in a pharmaceutically acceptable carrier, in amounts and for periods which will vary depending upon the nature of the particular disease, its severity and the patient's overall condition.

C. Delivery Devices

The present invention further includes drug delivery devices comprising a raf antisense oligonucleotide. The devices of the present invention may further comprise one or more additional therapeutic agents. The drug delivery devices of the present invention are particularly well-suited for the administration and controlled release of therapeutic agents over a prolonged time-course. Accordingly, they offer significant advantages over other methods of administration, such as, e.g., ocular injection or systemic intravenous delivery, since they require only a single injection or surgical implantation of the drug delivery device comprising a raf antisense oligonucleotide, as compared to the multiple intraocular or periocular injections of raf antisense oligonucleotides or multiple intravenous administrations required when the raf antisense oligonucleotide is not in a drug delivery device. Drug delivery devices according to the present invention are typically biocompatible.

Drug delivery devices according to the present invention include microcapsules. In certain embodiment, an oligonucleotide of the present invention is entrapped in a microcapsule prepared, for example, by coacervation techniques or by interfacial polymerization, for example, hydroxymethylcellulose or gelatin-microcapsules and poly-(methylmethacylate) microcapsules, respectively; in colloidal drug delivery systems (for example, liposomes, niosomes, albumin microspheres, microemulsions, nano-particles and nanocapsules) or in macroemulsions. Such techniques are disclosed in Remington's Pharmaceutical Sciences 16th edition, Osol, A. Ed. (1980). Other microcapsules include, e.g., nanoparticles and microparticles.

In one embodiment, a drug delivery device of the present invention comprises a sustained-release preparation of a raf antisense oligonucleotide. Suitable examples of sustained-release preparations include semipermeable matrices of solid hydrophobic polymers containing the antagonist, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and .gamma. ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), biocompatible polymers, and poly-D-(−)-3-hydroxybutyric acid.

In additional embodiments, drug delivery devices according to the present invention comprise a biocompatible matrix. Biocompatible matrices of the present invention may comprise, e.g., collagen, metal, hydroxyapatite, bioglass, aluminate, bioceramic materials, and purified proteins.

In one particular embodiment, the drug delivery device comprises Atrigel™ (QLT, Inc., Vancouver, B.C.). The Atrigel® drug delivery system consists of biodegradable polymers dissolved in biocompatible carriers. Pharmaceuticals may be blended into this liquid delivery system at the time of manufacturing or, depending upon the product, may be added later by the physician at the time of use. When the liquid product is injected into the subcutaneous space through a small gauge needle or placed into accessible tissue sites through a cannula, water in the tissue fluids causes the polymer to precipitate and trap the drug in a solid implant. The drug encapsulated within the implant is then released in a controlled manner as the polymer matrix biodegrades with time.

In another embodiment, the drug delivery device comprises an erodable polylacticglycolic acid (PLGA) matrix, such as that used in the Posurdex® system (Allergan, Irvine, Calif.). Posurdex® is a bioerodable extended release implant that delivers dexamethasone to the targeted disease site at the posterior-segment of the eye.

A delivery system employed in other embodiments of the present invention is ocular iontophoresis, such as, e.g., the OcuPhor™ system developed by Iomed (Salt Lake City, Utah), the Optis system developed by Eyegate Pharma (Paris, France) and others developed by Aciont, Inc (Salt Lake City, Utah). Such systems could be readily modified for use in delivering the therapeutic compositions and ocular drugs described herein.

For treatment of ocular diseases, topical application of drugs with solutions, suspensions, gels, and ointments has been a main route for their administration. However, drug clearance from the ocular surface is so rapid that therapeutic levels cannot be maintained for sustained periods without frequent applications. Absorption enhancers or viscosity enhancers has been investigated to increase the drug availability. Topically applied drug usually penetrates the cornea and reaches the anterior segments. However, the drug hardly reaches the posterior segments after topical instillation. Due to the poor penetration of the drugs to the posterior segments after topical instillation, diseases affecting the posterior segments of the eye are difficult to treat by this route of administration. Direct injection of drugs into the vitreous cavity is sometimes employed to achieve high drug concentration in the vitreous and the retina. However, repeated injections are needed to maintain a drug concentration at the effective range for a certain period of time since a half-life of drugs in the vitreous is relatively short. Repeated injections cause patients discomfort and may lead to complications such as vitreous hemorrhage, infection, and lens or retinal injury. Systemic administration of drugs has been used to treat some vitreoretinal diseases. But previous studies reported a very small amount of drugs could reach the eye after systemic administration. A large systemic amount is required to obtain a therapeutic level of drug concentration in the eye. In addition, the blood-retinal barrier that is located at the level of retinal vascular endothelial cells and retinal pigment epithelium inhibits an entry of certain drugs from systemic circulation into the retinal tissue. For the above reasons, drug delivery to the posterior segments of the eye has been challenging for both clinicians and basic scientists.

In certain embodiments, the present invention alleviates these problems by providing a drug delivery device suitable for ocular administration of a raf antisense oligonucleotide, alone or in combination with one or more additional therapeutic agents.

One example of a suitable ocular drug delivery device is a polymeric device that has a reservoir containing the drug which is surrounded with semi-permeable membrane. The polymeric semi-permeable membrane allows a sustained release of the drug for a couple of years. The device is implanted in the vitreous through the pars plana. Another polymer-based device of the present invention is a mucoadhesive polymer.

In another embodiment, a drug delivery device of the present invention comprises a polymer. Polymers such as poly(lactic acid) or poly(glycolic acid) undergo hydrolytic degradation in the body and become monomers of lactic acid or glycolic acid. These monomers can be metabolized and eliminated from the tissues. It is possible to incorporate drugs in the matrix of these polymers. The polymer containing the drug releases the drug for a sustained period and undergoes degradation simultaneously. These polymers have been used as materials of absorbable surgical sutures for many years and proved to be safe and biocompatible. Feasibility of delivering drugs to the retina and vitreous as well as the subconjunctival space using the microspheres of biodegradable polymers has been reported. A suspension of the microsphers can be injected through a fine needle. These microspheres maintain the drug concentration for a therapeutic level for several weeks to several months after the single administration. In one particular embodiment, a polymer comprising a raf antisense oligonucleotide is attached to a tissue in the eye called the conjunctiva.

The sodium salt of hyaluronic acid (SH) is a high molecular weight biological polymer, made of repeating disaccharide units of glucoronic acid and N-acetyl-b-glucosamine. In the eye, SH is present in the vitreous body and, in lower concentrations, the aqueous humor.

Another example of a drug delivery device of the present invention is an adjustable transscleral delivery device, which, in one embodiment, is an 8-by-6 millimeter scleral drug delivery device, 4 mm thick, made out of the same material used for scleral buckles.

In another embodiment, a drug delivery device of the present invention is a silicone intravitreal insert.

In one embodiment, a drug delivery device of the present invention is a calcium-alginate insert that can be placed in the conjunctival cul-de-sac to deliver drugs for a few days at a time.

In another embodiment, the drug delivery device is an implant, to be placed underneath LASIK flaps. The insert typically degrades in about four weeks.

In one embodiment, the drug delivery device of the present invention comprises a raf antisense oligonucleotide expression construct encapsulated in a cell. A French biotechnology firm, Neurotech, engineers cells that produce a therapeutic protein, then encases them in a protective carrier that can be implanted in the vitreous. It reports success at using this approach with neurotrophic factor, preventing retinal degeneration in animal models.

In another embodiment, the drug delivery device comprises calcium phosphate nanoparticles comprising a raf antisense oligonucleotide of the present invention.

Another embodiment contemplates the use of penetration enhancers such as cytochalasin B.

Hydrophobic or hydrophilic polymers shaped into a sheet, disc, rod, plug, or a larger device can be implanted into the subretinal space, intrascleral space, vitreous space, peribulbar space, or at the pars plana. Many researchers suggest the feasibility of these implants to treat AMD.

Other examples of drug delivery devices according to the present invention include drug-eluting stents, silicone microspheres loaded with cytostatic drugs in the established endotamponade silicone oil and the newly developed perfluorhexyloctane.

The InnoRx sustained release system utilizes SurModics' Bravo™ drug delivery polymer coating and a non-biodegradable implantable coil designed for sustained release of therapeutics into the posterior chamber of the eye. As such, it is ideally suited for site-specific treatment of diseases such as diabetic macular edema (DME) and age-related macular degeneration (AMD). The platform is implanted through a minimally invasive pars plana needlestick less than 0.5 mm in diameter. The unique helical design maximizes the surface area available for drug delivery, and ensures secure anchoring of the implant against the sclera, keeping it out of the visual field and facilitating retrieval. The thin cap is designed to reside under the conjunctival membrane of the eye. The implant is capable of providing long-term drug delivery, thus replacing frequent intraocular injections, the current standard of care.

Other drug delivery devices comprise miniature implantable pumps that deliver fluid to the eye for an extended period.

Ocular drug delivery systems may be evaluated by various methods, including in vitro evaluation methods, e.g., bottle method, diffusion method, modified rotating basket method, modified rotating paddle apparatus, and flow through devices, and in vivo animal models. Particularly suitable drug delivery devices of the present invention provide prolonged delivery of the raf antisense oligonucleotide and/or one or more additional therapeutic agents as compared to the duration of delivery in the absence of the drug delivery device. In preferred embodiments, a drug delivery device increases the pharmacokinetics, e.g., half-life, of a raf antisense oligonucleotide by at least two-fold, at least five-fold, or at least ten-fold as compared to in the absence of the drug delivery device.

D. Methods of Treating or Preventing Cancer and Ocular Diseases and Disorders

The present invention further provides method of regulating cell growth, treating or preventing tumors, reducing or preventing cancer growth and metastasis, and inhibiting neovascularization, comprising providing to a cell or patient a raf antisense oligonucleotide in combination with one or more additional therapeutic agents. In certain aspects of the present invention, the raf antisense oligonucleotide and/or one or more of the additional therapeutic agents are provided to a patient in a drug delivery device, typically to achieve prolonged delivery over a relatively long time period. In certain embodiments, this time period is at least one week, at least two weeks, at least three weeks, at least one month, at least two months, at least three months, at least four months, at least six months, at least nine months, or at least one year.

As described herein, oligonucleotides targeted to portions of the c-raf mRNA are particularly useful for inhibiting raf expression and for interfering with cell hyperproliferation. Methods for inhibiting c-raf expression using antisense oligonucleotides are, likewise, useful for interfering with cell hyperproliferation and neovascularization. In certain embodiments of the methods of the invention, tissues or cells are contacted with oligonucleotides. In the context of this invention, to “contact” tissues or cells with an oligonucleotide or oligonucleotides means to add the oligonucleotide(s), usually in a liquid carrier, pharmaceutical composition, and/or drug delivery device, to a cell suspension or tissue sample, either in vitro or ex vivo, or to administer the oligonucleotide(s) to cells or tissues within an animal, again, typically in a pharmaceutical formulation or drug delivery device.

The methods of the present invention may be used to treat any disease or disorder that benefits from reduced raf expression. In particular embodiments, the disease or disorder is associated with cellular hyperproliferation or pathogenic angiogenesis. Accordingly, the present invention may be used to treat or prevent a variety of diseases and disorders, including, but not limited to, cancer, e.g., solid and liquid tumors, tumor cell metastasis, autoimmune diseases and disorders, arthritis, and diseases associated with neovascularization.

In one embodiment, a method or drug delivery device of the present invention is used to treat an ocular disease or disorder. An “ocular disorder” herein is a disease or disorder involving the eye. Ocular diseases include, but are not limited to, various proliferative diseases like proliferative vitreoretinopathy, infectious diseases as endophthalmitis, inflammatory diseases such as uveitis, vascular diseases such as diabetic retinopathy, cystoid macular edema, corneal neovascularization, age-related amcular degeneration, retinal vein or arterial occlusion, and degenerative disorders such as age-related macular degeneration and retinitis pigmentosa. In addition, diseases like glaucoma or optic neuritis might be potentially treated with the effective delivery of neuroprotective agents to the retina and optic nerve.

Age-related macular degeneration (AMD) is a leading cause of severe visual loss in the elderly population. The exudative form of AMD is characterized by choroidal neovascularization and retinal pigment epithelial cell detachment. Because choroidal neovascularization is associated with a dramatic worsening in prognosis, the raf antisense oligonucleotides of the present invention are especially useful in reducing the severity of AMD. The progress of this therapy is easily monitored by conventional techniques and assays, including, for example, radiographic tumor imaging.

Macular degeneration affects between five and ten million patients in the United States, and it is the leading cause of blindness worldwide. Macular degeneration affects central vision and causes the loss of photoreceptor cells in the central part of retina called the macula. Macular degeneration can be classified into two types: dry type and wet type. The dry form is more common than the wet, with about 90% of age-related macular degeneration (ARMD) patients diagnosed with the dry form. The wet form of the disease and geographic atrophy, which is the end-stage phenotype of dry ARMD, lead to more serious vision loss. All patients who develop wet form ARMD previously had dry form ARMD for a prolonged period of time. The exact causes of age-related macular degeneration are still unknown. The dry form of ARMD may result from the aging and thinning of macular tissues and from deposition of pigment in the macula. In wet ARMD, new blood vessels grow beneath the retina and leak blood and fluid. This leakage causes the retinal cells to die, creating blind spots in central vision.

For the vast majority of patients who have the dry form of macular degeneration, no treatment is available. Because the dry form precedes development of the wet form of macular degeneration, intervention in disease progression of the dry form could benefit patients that presently have dry form and may delay or prevent development of the wet form.

Diabetic retinopathy occurs when diabetes damages blood vessels inside the retina. Non-proliferative retinopathy is a common, usually mild form that generally does not interfere with vision. Abnormalities are limited to the retina, and vision is impaired only if the macula is involved. If left untreated it can progress to proliferative retinopathy, the more serious form of diabetic retinopathy. Proliferative retinopathy occurs when new blood vessels proliferate in and around the retina. Consequently, bleeding into the vitreous, swelling of the retina, and/or retinal detachment may occur, leading to blindness.

In other embodiments, the invention provides a method of treating or preventing any of various tumors (cancers) and non-neoplastic diseases and disorders. Neoplasms and related conditions that are amenable to treatment include breast carcinomas, lung carcinomas, gastric carcinomas, esophageal carcinomas, colorectal carcinomas, liver carcinomas, ovarian carcinomas, thecomas, arrhenoblastomas, cervical carcinomas, endometrial carcinoma, endometrial hyperplasia, endometriosis, fibrosarcomas, choriocarcinoma, head and neck cancer, nasopharyngeal carcinoma, laryngeal carcinomas, hepatoblastoma, Kaposi's sarcoma, melanoma, skin carcinomas, hemangioma, cavernous hemangioma, hemangioblastoma, pancreas carcinomas, retinoblastoma, astrocytoma, glioblastoma, Schwannoma, oligodendroglioma, medulloblastoma, neuroblastomas, rhabdomyosarcoma, osteogenic sarcoma, leiomyosarcomas, urinary tract carcinomas, thyroid carcinomas, Wilm's tumor, renal cell carcinoma, prostate carcinoma, abnormal vascular proliferation associated with phakomatoses, edema (such as that associated with brain tumors), and Meigs' syndrome.

In addition, a raf antisense oligonucleotide in conjunction with a drug delivery device is useful in the delivery of drug for the treatment of ovarian cancer by means of an intraperitoneal perfusion of the drug in a delivery system that would extend the resident time of the drug in the peritoneal cavity to treat metastasis present in the peritoneal cavity.

According to various embodiments of the methods of the present invention, the raf antisense oligonucleotide is delivered prior to, concurrently with, or after delivery of the one or more additional therapeutic agents used in the combination therapy. One or more of the raf antisense oligonucleotide and one or more additional therapeutic agents may be delivered using a single drug delivery devices, or, alternatively, they may be delivered in separate drug delivery devices. Of course, in particular embodiments, none, one or some of the therapeutic agents are delivered using a drug delivery device.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method of treating or preventing an ocular disease or disorder comprising administering to a patient or cells thereof a therapeutically effective amount of an oligonucleotide 8 to 50 nucleotides in length which is targeted to mRNA encoding human c-raf (SEQ ID NO: 28), wherein said oligonucleotide inhibits expression of human c-raf, in combination with an ocular agent.

2. The method of claim 1 wherein said ocular disease or disorder is selected from the group consisting of: age related macular degeneration, diabetic retinopathy, diabetic macular edema, cystoid macular edema, and corneal neovascularization.

3. The method of claim 1 wherein said ocular agent is selected from the group consisting of: Macugen™, Avastin™, Sirna-027™, Cand5™, VEGF-TRAP, and Lucentis™, Visudyne™, Retaane™, Envizon™, Combretastatin™, AdPEDF, CAND5, kringle5, ganciclovir, ketotifen, verteporfin, pegaptanib, anecortare, dexamethasone, raibizumab, fluocinolone acetonide, and lerdelimumab.

4. A method of treating or preventing growth or metastasis of a tumor comprising administering to a patient or cells thereof a therapeutically effective amount of an oligonucleotide 8 to 50 nucleotides in length which is targeted to mRNA encoding human c-raf (SEQ ID NO: 28), wherein said oligonucleotide inhibits expression of human c-raf, in combination with an antitumor agent.

5. The method of claim 4 wherein said tumor is an ovarian cancer.

6. The method of claim 4 wherein said antitumor agent is a chemotherapeutic agent.

7. A drug delivery device comprising a biocompatible carrier and a therapeutically effective amount of an oligonucleotide 8 to 50 nucleotides in length which is targeted to mRNA encoding human c-raf (SEQ ID NO: 28), wherein said oligonucleotide inhibits expression of human c-raf.

8. The drug delivery device of claim 7 wherein said biocompatible carrier comprises a biocompatible matrix.

9. The drug delivery device of claim 8, wherein said biocompatible matrix is selected from the group consisting of: a polymer, collagen, metal, hydroxyapatite, bioglass, aluminate, bioceramic materials, and purified proteins.

10. The drug delivery device of claim 7 wherein said biocompatible carrier comprises a microcapsule.

11. The method of any of claims 1 or 4 or the device of claim 7, wherein the oligonucleotide is a full phosphorothioate analog consisting of the sequence, TCCCGCCTGTGACATGCATT (SEQ ID NO:8), with 2′-O-methoxyethyl substitutions at positions 1-6 and 15-20, and wherein residues 7-14 are unmodified 2′-deoxy.


SEQUENCE LISTING (28 sequences)

SEQ ID NO: 1 - DNA, 20 bp, Artificial Sequence
  Synthetic oligonucleotide
tgaaggtgagctggagccat

SEQ ID NO: 2 - DNA, 20 bp, Artificial Sequence
  Synthetic oligonucleotide
gctccattgatgcagcttaa

SEQ ID NO: 3 - DNA, 20 bp, Artificial Sequence
  Synthetic oligonucleotide
ccctgtatgtgctccattga

SEQ ID NO: 4 - DNA, 20 bp, Artificial Sequence
  Synthetic oligonucleotide
ggtgcaaagtcaactagaag

SEQ ID NO: 5 - DNA, 20 bp, Artificial Sequence
  Synthetic oligonucleotide
attcttaaacctgagggagc

SEQ ID NO: 6 - DNA, 20 bp, Artificial Sequence
  Synthetic oligonucleotide
gatgcagcttaaacaattct

SEQ ID NO: 7 - DNA, 20 bp, Artificial Sequence
  Synthetic oligonucleotide
cagcactgcaaatggcttcc

SEQ ID NO: 8 - DNA, 20 bp, Artificial Sequence
  Synthetic oligonucleotide
tcccgcctgtgacatgcatt

SEQ ID NO: 9 - DNA, 20 bp, Artificial Sequence
  Synthetic oligonucleotide
gccgagtgccttgcctggaa

SEQ ID NO: 10 - DNA, 20 bp, Artificial Sequence
  Synthetic oligonucleotide
agagatgcagctggagccat

SEQ ID NO: 11 - DNA, 20 bp, Artificial Sequence
  Synthetic oligonucleotide
aggtgaaggcctggagccat

SEQ ID NO: 12 - DNA, 20 bp, Artificial Sequence
  Synthetic oligonucleotide
gtctggcgctgcaccactct

SEQ ID NO: 13 - DNA, 20 bp, Artificial Sequence
  Synthetic oligonucleotide
ctgatttccaaaatcccatg

SEQ ID NO: 14 - DNA, 20 bp, Artificial Sequence
  Synthetic oligonucleotide
ctgggctgtttggtgcctta

SEQ ID NO: 15 - DNA, 20 bp, Artificial Sequence
  Synthetic oligonucleotide
tcagggctggactgcctgct

SEQ ID NO: 16 - DNA, 20 bp, Artificial Sequence
  Synthetic oligonucleotide
ggtgagggagcgggaggcgg

SEQ ID NO: 17 - DNA, 20 bp, Artificial Sequence
  Synthetic oligonucleotide
cgctcctcctccccgcggcg

SEQ ID NO: 18 - DNA, 20 bp, Artificial Sequence
  Synthetic oligonucleotide
ttcggcggcagcttctcgcc

SEQ ID NO: 19 - DNA, 20 bp, Artificial Sequence
  Synthetic oligonucleotide
gccgccccaacgtcctgtcg

SEQ ID NO: 20 - DNA, 20 bp, Artificial Sequence
  Synthetic oligonucleotide
tcctcctccccgcggcgggt

SEQ ID NO: 21 - DNA, 20 bp, Artificial Sequence
  Synthetic oligonucleotide
ctcgcccgctcctcctcccc

SEQ ID NO: 22 - DNA, 20 bp, Artificial Sequence
  Synthetic oligonucleotide
ctggcttctcctcctcccct

SEQ ID NO: 23 - DNA, 20 bp, Artificial Sequence
  Synthetic oligonucleotide
cgggaggcggtcacattcgg

SEQ ID NO: 24 - DNA, 20 bp, Artificial Sequence
  Synthetic oligonucleotide
tctggcgctgcaccactctc

SEQ ID NO: 25 - DNA, 20 bp, Artificial Sequence
  Synthetic oligonucleotide
ttctcgcccgctcctcctcc

SEQ ID NO: 26 - DNA, 20 bp, Artificial Sequence
  Synthetic oligonucleotide
ttctcctcctcccctggcag

SEQ ID NO: 27 - DNA, 20 bp, Artificial Sequence
  Synthetic oligonucleotide
cctgctggcttctcctcctc

SEQ ID NO: 28 - DNA, 2977 bp, Homo sapiens
ccgaatgtgaccgcctcccgctccctcacccgccgcggggaggaggagcgggcgagaagctgccgccgaacgacaggacgttggggcggcctggctccctcaggtttaagaattgtttaagctgcatcaatggagcacatacagggagcttggaagacgatcagcaatggttttggattcaaagatgccgtgtttgatggctccagctgcatctctcctacaatagttcagcagtttggctatcagcgccgggcatcagatgatggcaaactcacagatccttctaagacaagcaacactatccgtgttttcttgccgaacaagcaaagaacagtggtcaatgtgcgaaatggaatgagcttgcatgactgccttatgaaagcactcaaggtgaggggcctgcaaccagagtgctgtgcagtgttcagacttctccacgaacacaaaggtaaaaaagcacgcttagattggaatactgatgctgcgtctttgattggagaagaacttcaagtagatttcctggatcatgttcccctcacaacacacaactttgctcggaagacgttcctgaagcttgccttctgtgacatctgtcagaaattcctgctcaatggatttcgatgtcagacttgtggctacaaatttcatgagcactgtagcaccaaagtacctactatgtgtgtggactggagtaacatcagacaactcttattgtttccaaattccactattggtgatagtggagtcccagcactaccttctttgactatgcgtcgtatgcgagagtctgtttccaggatgcctgttagttctcagcacagatattctacacctcacgccttcacctttaacacctccagtccctcatctgaaggttccctctcccagaggcagaggtcgacatccacacctaatgtccacatggtcagcaccacgctgcctgtggacagcaggatgattgaggatgcaattcgaagtcacagcgaatcagcctcaccttcagccctgtccagtagccccaacaatctgagcccaacaggctggtcacagccgaaaacccccgtgccagcacaaagagagcgggcaccagtatctgggacccaggagaaaaacaaaattaggcctcgtggacagagagattcaagctattattgggaaatagaagccagtgaagtgatgctgtccactcggattgggtcaggctcttttggaactgtttataagggtaaatggcacggagatgttgcagtaaagatcctaaaggttgtcgacccaaccccagagcaattccaggccttcaggaatgaggtggctgttctgcgcaaaacacggcatgtgaacattctgcttttcatggggtacatgacaaaggacaacctggcaattgtgacccagtggtgcgagggcagcagcctctacaaacacctgcatgtccaggagaccaagtttcagatgttccagctaattgacattgcccggcagacggctcagggaatggactatttgcatgcaaagaacatcatccatagagacatgaaatccaacaatatatttctccatgaaggcttaacagtgaaaattggagattttggtttggcaacagtaaagtcacgctggagtggttctcagcaggttgaacaacctactggctctgtcctctggatggccccagaggtgatccgaatgcaggataacaacccattcagtttccagtcggatgtctactcctatggcatcgtattgtatgaactgatgacgggggagcttccttattctcacatcaacaaccgagatcagatcatcttcatggtgggccgaggatatgcctccccagatcttagtaagctatataagaactgccccaaagcaatgaagaggctggtagctgactgtgtgaagaaagtaaaggaagagaggcctctttttccccagatcctgtcttccattgagctgctccaacactctctaccgaagatcaaccggagcgcttccgagccatccttgcatcgggcagcccacactgaggatatcaatgcttgcacgctgaccacgtccccgaggctgcctgtcttctagttgactttgcacctgtcttcaggctgccaggggaggaggagaagccagcaggcaccacttttctgctccctttctccagaggcagaacacatgttttcagagaagctctgctaaggaccttctagactgctcacagggccttaacttcatgttgccttcttttctatccctttgggccctgggagaaggaagccatttgcagtgctggtgtgtcctgctccctccccacattccccatgctcaaggcccagccttctgtagatgcgcaagtggatgttgatggtagtacaaaaagcaggggcccagccccagctgttggctacatgagtatttagaggaagtaaggtagcaggcagtccagccctgatgtggagacacatgggattttggaaatcagcttctggaggaatgcatgtcacaggcgggactttcttcagagagtggtgcagcgccagacattttgcacataaggcaccaaacagcccaggactgccgagactctggccgcccgaaggagcctgctttggtactatggaacttttcttaggggacacgtcctcctttcacagcttctaaggtgtccagtgcattgggatggttttccaggcaaggcactcggccaatccgcatctcagccctctcaggagcagtcttccatcatgctgaattttgtcctccaggagctgcccctatggggcgggccgcagggccagcctgtttctctaacaaacaaacaaacaaacagccttgtttctctagtcacatcatgtgtatacaaggaagccaggaatacaggttttcttgatgatttgggttttaattttgtttttattgcacctgacaaaatacagttatctgatggtccctcaattatgttattttaataaaataaattaaattt